(19) United States
(12) Patent Application Publication
(10) Pub. No.: US 2017/0319782 A1
(43) Pub. Date: Nov. 9, 2017

(54) Device for Maintaining the Arterial Pressure in a Recommended Target

(71) Applicants: Université D'Angers, Angers (FR); Centre Hospitalier Universitaire D'Angers (C.H.U. D'Angers), Angers (FR)

(72) Inventor: Pierre Asfar, Angers (FR)

(21) Appl. No.: 15/500,325

(22) PCT Filed: Jul. 27, 2015
(86) PCT No.: PCT/FR2015/052083

§ 371 Date: Jul. 20, 2017

(30) Foreign Application Priority Data
Jul. 29, 2014 (FR) 1401761

(51) Int. Cl. A61M 5/172; A61M 5/168; A61M 5/142

(52) U.S. Cl. CPC A61M 5/1723; A61M 5/16804; A61M 2205/583; A61M 2205/581; A61M 2205/50; A61M 2205/3303; A61M 2205/18; A61M 2005/1726; A61M 2005/14208; A61M 2230/06; A61M 2205/3344

(57) ABSTRACT

The invention relates to a method of determining an infusion rate in a medical device to aid with administration of a substance as a function of a physiological parameter and a device adapted to implementation of the method, and a computer program implementing the method.

### BACKGROUND

The invention relates to the domain of medical equipment and particularly to a device for aid with proportioning and administration of at least one medicinal substance depending on at least one physiological parameter of a patient.

### SUMMARY

Testing the arterial blood pressure of a treated patient combines vascular filling and administration of catecholamines in proportions that depend on the symptoms present.

Hemodynamic management of patients in a state of shock, and particularly in a state of septic shock, is based on recommendations issued by the Surviving Sepsis Campaign (SSC). The SSC is an independent entity with the objective of fighting against sepsis (severe infection of the blood), with the support of several resuscitation companies in Europe and in the United States. It is committed to reducing mortality due to severe sepsis and septic shock throughout the world.

Recommendations issued by the SSC stipulate that vascular filling must be done based on measured values of the central venous blood pressure and an invasively measured target average arterial blood pressure. The target average arterial pressure thus recommended is more than 65 mmHg.

Although doctors issue instructions conforming with these recommendations, in practice these recommendations are implemented in a manner such that they cannot always be respected sufficiently precisely. There are sometimes large differences for different reasons. There is also a phenomenon of episodes characterised by arterial pressure being above or below recommendations during short periods. Medical personnel are not always sufficiently available to react taking account of these variations over short periods because this would mean that the medical person would always need to be close to the monitoring equipment.

A manual adjustment of the infusion rate of catecholamines at the specified level cannot always be made precisely due to the very high variability of the average arterial pressure. Furthermore, a manual adjustment is usually made once or twice every hour based on a monitoring result that is representative of an average arterial pressure over a very long time. The result is upwards and downwards deviations of the average arterial pressure that can be harmful to the health of patients and/or lead to secondary effects that could have been avoided by keeping the blood pressure closer to the target.

If the average arterial pressure, that is the infusion pressure into organs, becomes too low, there is a risk of hypo-infusion and an ischemic risk that could lead to or aggravate a failure of one or several organs.

If the average arterial pressure becomes too high due to an excessive injection of catecholamines, the result is large vasoconstriction also accompanied by an ischemic risk and a risk of heart rate problems.

A test campaign referred to as SEPSISPAM, consisting of evaluating the effect of two arterial pressure levels on the survival of patients in a state of septic shock and a corresponding article, demonstrate and describe these aspects.

A random and multicentric clinical test in Canada aimed at optimising vasopressors in hypotension, called OVATION, demonstrated the same limitations concerning applied control over the arterial pressure of a patient.

Consequently, the quantity of catecholamines useful for keeping the blood pressure within a recommended target prescribed by a doctor and no more, should be administrated, avoiding harmful pressure variations and particularly fast variations that cannot by shown up be existing means.

However the average arterial pressure is a variable with a fast adaptation (a few minutes) and the infusion rate is adapted by nursing personnel responsible for a patient who usually modify it only once or twice per hour.

The “An electronic, negative feedback device to control arterial pressure” document (Benet J. Pardini, Donald D. Lund, Robert D. Wurster, and Roger H. Anderson, received 26 May 1986; accepted in final form 27 Aug. 1987) describes a unit for controlling the arterial pressure of a rat to keep it between 100 and 200 mmHg, combining the delivery of an active substance through an infusion set and a slaving loop.

Document WO2012/036636 A1 (Singapore Health Sery Pte Ltd; Sia Tiong-Heng Alex) describes a system for the administration of medicinal substances by means of a plurality of infusion sets. The system controls the different infusion pumps as a function of the arterial pressure. U.S. Pat. No. 7,097,618 B1 (Benditt David G et al.—Aug. 29, 2006) describes a method and devices to detect inadequate administration by the measurement of a parameter other than the heart rate, for example such as the arterial pressure in addition to or instead of measuring the heart rate. Document WO 01/83007 A2 (Aspect Medical System Inc—8 Nov. 2001) describes a slaved method and device for maintaining an effect on a patient. Document U.S. Pat. No. 4,080,966 A (McNally Robert et al. 28 Mar. 1978) describes a method of slaving the arterial pressure of mammals by the use of a slaved system comparing a measured arterial pressure and a targeted arterial pressure.

Existing solutions have disadvantages, particularly concerning the symptomatic treatment of patients in a state of shock.

The invention improves the state of the art by disclosing a method and an associated calculation device to calculate the infusion rate of catecholamines as a function of at least one physiological parameter of a treated patient, and for example to program an infusion set using information supplied by a module that determines an infusion rate, as a function of a physiological parameter for example such as the value of the average arterial pressure of a treated patient. The physiological parameter considered may also for example be a marker in the blood, information representative of the condition of a patient's heart, information representative of a patient's breathing condition, or information representative of the hepatic condition of a patient.

Throughout the remainder of this document, the infusion rate is the quantity to be administrated into the patient's venous system during a predetermined time interval, or the average flow of active substance administrated during a defined time interval. For example, the infusion rate can be expressed as a quantity of substance per minute or per hour, independently of the type of infusion mode or infusion set used. Also, and in the case in which an electric infusion set is used, for example, the infusion rate does not correspond directly to a non-null speed of an actuator included in the infusion set, but to the quantity of substance administrated over a defined time interval, however related to the activation profile of the infusion set used over this time interval.

The invention consists of a method of determining an administration rate (possibly in an infusion device), the administration rate being slaved as a function of at least one parameter, the method including a determination of a plurality of successive values of the parameter, a definition of the infusion rate adapted to delivering a substance as a function of at least a part of the successive values, the method includes:

a determination of a first average value of at least a first part of successive values, the first part of the successive values being determined during a first predetermined time interval,

a determination of a second average value of at least a second part of successive values, the second part of successive values being determined during a second predetermined time interval, the second time interval having a duration shorter than the duration of the first time interval,

a calculation of the administration rate as a function of at least one result of a comparison between the first average value and the second average value.

According to one embodiment of the invention, and if an infusion set is used, the infusion set is an electric infusion set.

According to one embodiment of the invention, the parameter is a physiological parameter such as the arterial pressure, the pulse or a marker determined when a blood sample is taken.

According to one embodiment of the invention, the at least one parameter is an average arterial pressure.

Advantageously, the administration rate varies in successive steps.

According to one embodiment of the invention, the method also includes a check on the consistency of successive determined values in comparison with a predefined standard arterial pressure curve.

Advantageously, operations to determine the first and second average values and the calculated values are determined iteratively by execution of successive cycles.

According to one embodiment of the invention, an upwards or downwards variation of the administration rate during two consecutive successive cycles causes a modification to the state of an indicator.

Advantageously, the indicator is associated with a visual and/or sound alarm being triggered.

According to one embodiment of the invention, a predetermined minimum duration separates two successive variations of the rate.

According to one embodiment of the invention, the second time interval is determined as a function of a heart pulse rate.

According to one embodiment of the invention, the administrated substance includes catecholamines.

Advantageously, in a context of symptomatic treatment of a septic shock, the second average value is between lower and upper limits equal to 65 and 85 mmHg respectively, each value being configurable by the user.

According to one embodiment of the invention, a plurality of successive values of the parameter are determined using a device for recording an arterial pressure.

The invention also relates to a device for determining the infusion (administration) rate adapted to providing quantitative information of a substance over a given time interval (at an infusion rate), the infusion rate being slaved as a function of at least one parameter, the device comprising a module to determine a plurality of successive values of the parameter, a module to control the infusion rate as a function of at least part of the successive values, the device also comprising:

a module to determine a first average value of at least a first part of successive values, the first part of the successive values being determined during a first predetermined time interval,

a module to determine a second average value of at least a second part of said successive values, the second part of the successive values being determined during a second predetermined time interval, the second time interval having a duration shorter than the duration of the first time interval,

a calculator of the administration rate as a function of at least one result of a comparison between the first average value and the second average value.

The invention also relates to a computer program comprising program code instructions to run the steps in the method according to the invention when the program is run on a computer.

### BRIEF DESCRIPTION OF THE DRAWINGS

The invention will be better understood and other specific features and advantages will become clear after reading the following description given with reference to the appended drawings among which:

FIG. 1 illustrates a device according to a particular and non-limitative embodiment of the invention, making use of the method.

FIG. 2 represents successive arterial pressure measurement points useful for determining the infusion rate of a device to aid administration of a substance as a function of time, and according to a particular and non-limitative embodiment of the invention making use of the method.

FIG. 3 represents the measurement points in FIG. 2, as a function of time and a determined magnitude representative of the set value of an infusion rate, according to one particular and non-limitative embodiment of the invention.

FIG. 4 is a diagram illustrating the steps in the method of determining an infusion rate according to a particular and non-limitative embodiment of the invention.

### DETAILED DESCRIPTION

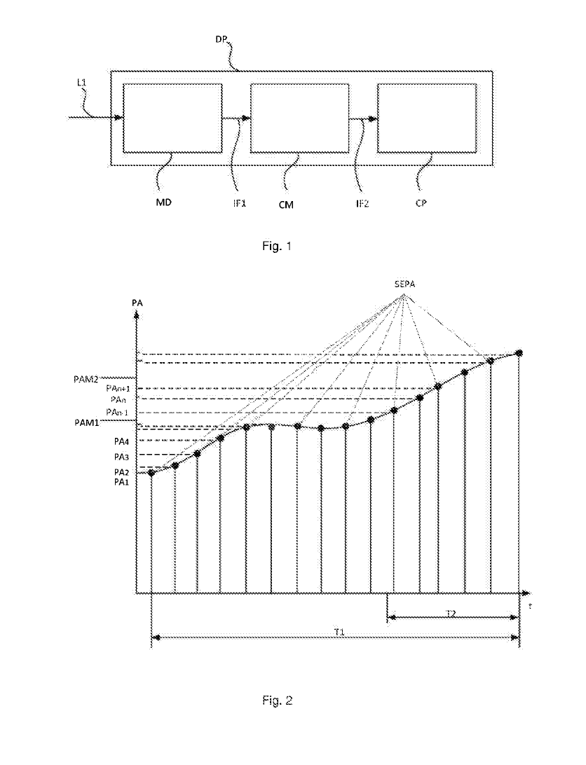

On FIG. 1, the modules represented are functional units that may or may not correspond to physically distinguishable units. For example, these modules or some of them are grouped together in a single component. On the other hand, in other embodiments, some modules are composed of separate physical entities.

FIG. 1 illustrates a device DP making use of the method according to a particular non-limitative embodiment of the invention. The device DP comprises a module MD to determine average arterial pressure values read through a link L1. The link L1 may include invasive or non-invasive means of measuring the arterial pressure of a patient. The module to determine the average values MD includes a module for storage of read (measured) values so that values can be processed after the measurements.

The device DP also includes a calculator CM adapted to determine an infusion rate as a function of data previously determined by the determination module MD. The device is configured so that it also calculates set values to be transmitted and useful for delivering a substance. The set value(s) depend(s) on the administration method and their determinations may depend on a configuration made using a man-machine interface. For example, the man machine interface is a console including a screen and a keyboard forming a part of the device DP or connected to the device DP.

According to the particular and non-limitative embodiment of the invention, the actuator used is a stepping motor controlled to move a syringe pusher device internal to the device DP. The controlled movement of the syringe pusher device leads to a determined infusion rate V1.

Average arterial pressures determined by the determination module MD are transmitted to the computer CM through a data interface IF1. The above-mentioned set values are transmitted by the calculator CM to the control module CP through the data interface IF2.

The determination module MD, the computer CM and the controller CP of the device DP each include a control unit configured to process input data and to determine output data. Each of these control units comprises a microprocessor, a non-volatile memory including executable instruction code and data useful for the corresponding processing, and a volatile portable memory functioning in particular as calculation registers. The microprocessors comprise at least one arithmetic and logical processing unit. The control units also include all elements well known to a computer science expert, for example such as interface buffers, signal shaping elements, clock circuits useful for clocking operations and synchronisation of the different elements, power supply interface circuits, power up initialisation circuits, etc. These different elements are not described herein since a description of the architecture of control units is routine and is not necessary to help understand the invention.

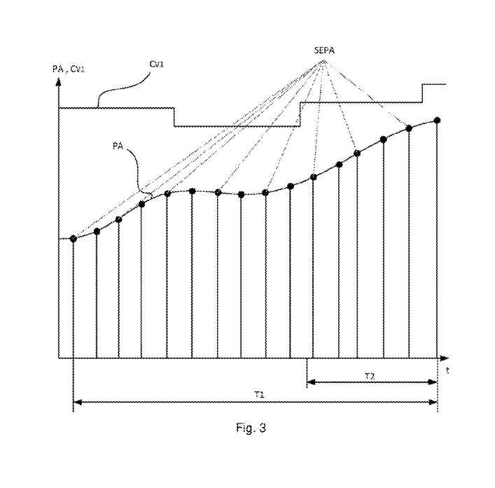

FIG. 2 represents successive measurement points of the arterial pressure useful for determining the infusion rate of the device DP shown in FIG. 1 as a function of time, and according to a particular and non-limitative embodiment of the invention making use of the method.

Values PAₙ of the arterial pressure of a patient are measured successively through the link L1, that may or may not be invasive for the patient. The sampling frequency of the arterial pressure is sufficiently high so that the module to determine average pressures can function on a subset SEPA of successively measured values PAₙ.

With the preferred embodiment of the invention, a first average PAM1 of successive values is calculated over a time interval equal to a number T1 of minutes elapsed continuously before this first calculation and an average PAM2 of successive values is calculated over a time interval equal to a number T2 of minutes elapsed continuously before this second calculation.

With the preferred embodiment, the duration of T1 is between 5 and 10 minutes and the duration of T2 is one minute.

Advantageously, time intervals T1 and T2 are preprogrammed with default values and can be reconfigured through a man-machine interface.

According to one preferred embodiment of the invention, the time interval T2 is a “sliding” time window that precedes the determination of a new value of the infusion rate V1.

According to one example embodiment, the computer CM performs an operation to compare the determined averages PAM1 and PAM2 and defines an infusion rate V1 as a function of the difference between PAM1 and PAM2. Thus, if the average value PAM1 is lower than the target pressure to be reached, and if PAM2 is less than PAM1, the computer CM determines a new value V1 of the infusion rate, higher than the previously calculated rate V1, based on prerecorded data, for example derived from calibration work based on the results of clinical research. Conversely, still in the case in which PAM1 is lower than the target pressure and if the determined average PAM2 is higher than the determined average PAM1, the computer CM determines a new infusion rate V1 lower than the previously calculated infusion rate.

Furthermore, if the average value PAM1 is higher than the target pressure to be reached, and if PAM2 is higher than PAM1, the computer CM determines a new value V1 of the infusion rate, lower than the previously calculated rate V1, based on prerecorded data, for example derived from calibration work based on the results of clinical research. And, still in the case in which PAM1 is higher than the target pressure and if the determined average PAM2 is lower than the determined average PAM1, the calculator CM determines a new infusion rate V1 higher than the previously calculated infusion rate but defined such that the average arterial pressure continues to reduce.

Advantageously, the comparative analysis of the averages PAM1 and PAM2 in order to determine the infusion rate V1 can prevent variations of the arterial pressure (sudden changes) that are considered to be potentially prejudicial to the patient.

Advantageously, the method according to the invention has a beneficial effect on the probability of occurrence of auricular fibrillation or renal insufficiency episodes in a context of a state of septic shock. The occurrence rate of auricular fibrillation episodes increases with increasing application of vasoconstrictors, for example such as catecholamines.

According to one embodiment of the invention, the calculator CM includes data representative of one or several standard variation curves of a patient's arterial pressure as a function of predefined parameters, such that it is possible to detect if measured pressure variations go outside these standard variation curves. Thus, if there is a non-conformity in the variation profile of the arterial pressure of a patient, a visual and/or sound alert can be triggered depending on the programmed configuration. Advantageously, the predefined parameters can be input or modified through a man-machine configuration interface. For example, these parameters are the age of the patient, his or her corpulence, information representative of his or her state of health (for example antecedents), whether or not predefined symptoms are present.

Advantageously, all information useful for performing the method of determining the infusion rate V1, including code instructions forming an algorithm implementing the method or the previously described standard curves, are included in a removable memory module that enables easy updates as a function of newly available research results specific to control over the arterial pressure of a patient or as a function of the patient's profile.

According to one embodiment of the invention, quality criteria of arterial pressure measurements are used to prevent parasites from being interpreted as variations of the arterial pressure. These criteria are based on the heart rate of the patient, assuming that one heart beat generates one arterial pressure wave shape.

Depending on the embodiment of the invention, high and low alarms indicate problems with slaving the patient's arterial pressure around the defined target value and successive slaving cycles leading to several successive increases or reductions in the infusion rate V1 cause a visual and/or sound alarm to be triggered. Advantageously, the number of successive cycles that lead to a variation of the infusion rate V1 in the same direction and that trigger an alarm can be configured. For example, this number is equal to two successive cycles.

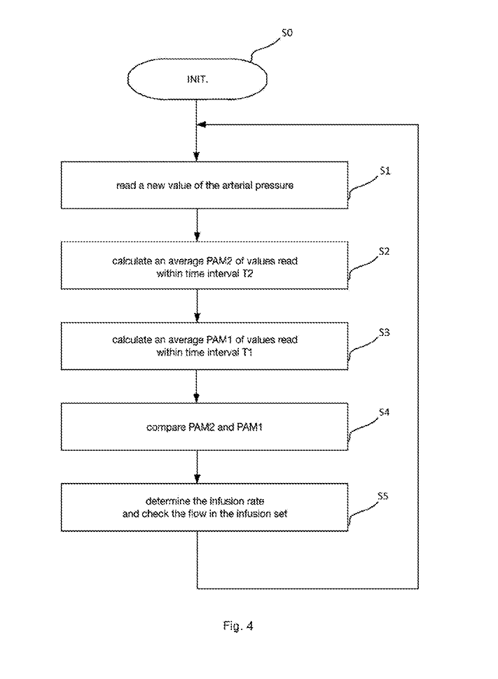

FIG. 3 shows measurement points of the average arterial pressure PA in FIG. 2 as a function of time t and a determined magnitude CV1 representative of the set value of the infusion rate V1 of the device DP, determined at least as a function of the comparison of the averages PAM1 and PAM2 determined over time intervals T1 and T2 respectively according to one particular and non-limitative embodiment of the invention.

FIG. 4 is a diagram illustrating steps in the method of determining an infusion rate according to a particular and non-limitative embodiment of the invention.

Step S0 is an initialisation step that precedes configuration of the device to aid administration of a substance DP in a nominal operating condition. Typically, this corresponds to a start up in which all modules MD, CM and CP are configured automatically and/or manually. At the end of this step, the device DP is available to deliver substance to the patient.

Step S1 corresponds to reading a new value PAn of the patient's arterial pressure, that will be taken into account to determine the average arterial pressures PAM1 and PAM2 over time intervals T1 and T2.

Step S2 corresponds to the calculation of the average PAM1 of values belonging to a subset SEPA of successively measured arterial pressures, by the calculation module CM. According to the preferred embodiment of the invention, the average PAM1 is determined over a time interval preceding the calculation and including several minutes, for example over a duration of 5 to 10 minutes.

Step S3 corresponds to the calculation of the average PAM2 of values belonging to a subset SEPA of successively measured arterial pressures, by the calculation module CM. The average PAM2 is determined over a one-minute time interval preceding the calculation.

Step S4 corresponds to the different analysis operations described above done by the calculator module CM, and particularly the comparison of the averages PAM1 and PAM2 transmitted from the determination MD to the computer module CM.

In this step, the calculator module outputs at least one set value CV1 signal (or data) representative of the determined infusion rate V1.

In step S5, the module controlling the administration aid device outputs data such that a quantity of substance is delivered to the patient corresponding to the infusion rate V1 determined in step S4. Step S5 also includes a configurable minimum waiting time before a new value of the arterial pressure PAn is read, and that corresponds to a “refractory period” useful for setting up a new average equilibrium of the arterial pressure resulting from holding or varying the infusion rate V1. For example, the refractory period may have a duration of a few minutes.

Time intervals T1 and T2 are called “sliding windows”.

In other words, and according to the non-limitative embodiment of the invention, the device DP implements a method of determining the slaved infusion rate V1 as a function of at least the measured average arterial pressure PA of the patient, the method including a determination of a plurality of successive values PAn of the average arterial pressure PA, control of the infusion rate infusion V1 adapted to the delivery of catecholamines as a function of at least a part SEPA of successively measured arterial pressure values, the method also including:

a determination of a first average value PAM1 of at least a first part of successively measured values, the first part of the successively measured values being determined during a first predetermined time interval T1, for example with a duration of several minutes (for example 5 to 10 minutes),

a determination of a second average value PAM2 of at least a second part of successively measured values, the second part of the successively measured values being determined during a second time interval T2, for example with a duration of one minute,

a calculation of the infusion rate V1 as a function of at least one result of a comparison between the first average value PAM1 and the second average value PAM2, and the value of the required target arterial pressure.

The definition of successive values V1 leads to a variation of the infusion rate in steps.

The method includes a check on the coherence of successive values PAn, by checking the coherence of the variation of the patient's arterial pressure against one or several predefined standard curves (threshold, typical variation, etc.).

These operations are performed iteratively by the execution of successive cycles, separated by a waiting time that is the refractory period.

In order to trigger an alarm, and if the device DP was configured to proceed in this manner, the method includes the possibility that an upwards or downwards variation of the infusion rate V1 during two consecutive successive cycles will change the state of an indicator internal to the device DP. This indicator is associated with a visual and/or sound alarm being triggered. Advantageously, the time interval T2 corresponding to the most recent “sliding” time minute is defined more precisely as a function of a previously determined heart rate of the patient.

Advantageously and in the context of a septic shock, the target arterial pressure to which the second average PAM2 should converge is between the lower and upper limits equal to 65 and 85 mm of Hg (millimetres height of mercury) respectively.

Successive values PAn of the arterial pressure value PAn are determined (measured) by means of an arterial pressure recording device like that already known to an expert in the field.

The device DP according to the preferred non-limitative embodiment of the invention is adapted to the delivery of catecholamines forming a substance C useful for maintaining a minimum arterial pressure level PA of the patient at an infusion rate V1, slaved as a function of the patient's arterial pressure PA. The device DP comprises a module for the determination of several successive measured values of the arterial pressure PAₙ, a module CM for control of its infusion rate V1 as a function of at least part of the measured arterial pressure values. The device DP also comprises the following according to the invention:

a module MD that determines a first average value PAM1 of at least a first part of successively measured values, the first part of the successively measured values PAn being determined during a first predetermined time interval T1, with a duration of several minutes,

a module MD that determines a second average value PAM2 of at least a second part of said successively measured values PAₙ, the second part of the successively measured values being determined during a second predetermined time interval T2, shorter than the first time interval T1, for example of the order of one minute,

a calculator CM of the infusion rate V1 as a function of at least one result of a comparison between the first average value PAM1 and said second average value PAM2.

Advantageously, the MD, CM and CP modules may all be included in the same control unit.

Advantageously and due to the fact the method according to the invention is used, the delivery of a vasoconstrictor product for example such as catecholamines is adjusted at a rate corresponding to the patient's physiological adjustment. The average reaction time to catecholamines is a few minutes.

Advantageously, the method according to the invention makes it possible to used only the required quantity of medicines (substance). This aspect is particularly important because the delivered substance is toxic to a certain extent, in addition to the benefit of holding the arterial pressure within an interval of predefined values.

According to one variant embodiment, the method comprises a set of processing done on signals available at the input to the device DP adapted to the quality of these signals. For example, it is advantageous to eliminate measurement parasites that might be present for example due to the detection of a cough, or patient movements. This is why it can be beneficial to check the coherence of a signal representing an average arterial pressure with a signal representative of a heart rate, for the same patient.

According to one embodiment of the invention, an algorithm used by an onboard software module in the device DP checks the synchronisation of at least one input signal representative of a patient's arterial pressure, with at least one other input signal representative of the heart rate of the same patient. For the purposes of this description, synchronisation refers to the fact that losses of signal variations considered at the input occur within the same time interval very much shorter than the period or “pseudo-period” of these signals.

Obviously, the invention is not limited to the embodiment described above but is applicable to any device using a method of determining an infusion rate as a function of a first average of a physiological parameter, for example such as the arterial pressure determined on a first time interval and a second average arterial pressure determined on a second time interval, at least one of these time intervals forming a continuous sliding time window at the end of which a new infusion rate is determined.

1.-9. (canceled)

10. A device adapted to determine an infusion rate that is a function of at least one parameter representative of an arterial pressure, the device comprising:
a module to determine a plurality of successive values of the parameter;
a module to control the infusion rate as a function of at least part of the successive values;
a module to determine a first average value of at least a first part of the successive values, the first part of the successive values being determined during a first predetermined time interval;
a module to determine a second average value of at least a second part of the successive values, the second part of said successive values being determined during a second predetermined time interval, the second predetermined time interval having a duration shorter than the duration of the first predetermined time interval, and being included in a same cycle to calculate the rate as the first time interval; and
a calculator to calculate an infusion rate as a function of at least one result of a comparison between the first average value and the second average value.

11. The device according to claim 10, wherein the rate varies in successive steps.

12. The device according to claim 10, further comprising a module to check the coherence of the successive values determined relative to a predefined standard arterial pressure curve.

13. The device according to claim 10, wherein operations to determine the first and second average values and the calculation are made iteratively by executing successive cycles.

14. The device according to claim 10 wherein an upward or downward variation of the rate during two consecutive successive cycles causes a change to a state of an indicator.

15. The device according to claim 14 wherein the indicator is associated with triggering of a visual and/or sound alarm.

16. The device according to claim 10 wherein a predetermined minimum duration separates two successive variations of the rate.

17. The device according to claim 10 wherein the second predetermined time interval is determined as a function of a heart rate.

18. The device according to claim 10 wherein the rate is defined such that the average second value is between about 65 mmHg and about 85 mmHg.
